(19) United States
(12) Patent Application Publication
(10) Pub. No.: US 2010/0297756 A1
(43) Pub. Date: Nov. 25, 2010

(54) MEANS FOR DELIVERY OF NUCLEIC ACIDS ACTIVE FOR GENE SILENCING USING SYNTHETIC POLYMERS

(76) Inventors: Abdennajj Adib, Illkirch (FR); Patrick Erbacher, Benfeld (FR); Fabrice Stock, Lingolsheim (FR); Nadia Hafdi, Illkirch (FR)

Correspondence Address: NIXON & VANDERHYE, PC, 901 NORTH GLEBE ROAD, 11TH FLOOR, ARLINGTON, VA, 22203, US

(21) Appl. No.: 12/735,031

(22) PCT Filed: Dec. 12, 2008
(86) PCT No.: PCT/IB2008/055256

§ 371 Date: Jun. 10, 2010

Related U.S. Application Data
(60) Provisional application No. 61/013,358, filed on Dec. 13, 2007.

(51) Int. Cl. C12N 5/071; C12N 15/79; C07C 229/00

(52) U.S. Cl. 435/366; 435/320.1; 562/433

(57) ABSTRACT

The invention relates to a composition useful as transfection agent, comprising polyamines modified by aromatic amino acids and small double-strand or single-strand RNA active for RNA interference.

The invention relates to means, compositions and methods, for efficient synthetic polymer-mediated delivery to eukaryotic cells in culture, in vivo or ex vivo of nucleic acids mediating gene silencing in cells, particularly small interfering RNA (designated as siRNA in the following description and the claims) providing RNA interference (RNAi) and optionally plasmid DNA.

RNA interference (RNAi) is a technology for gene silencing at the early gene function level, the mRNA (Fire et al, 1998). The principle is an extremely selective interaction of short RNA duplexes (siRNA; small interfering RNA) with a single target in the mRNA, providing sequence-specific mRNA degradation and thus inhibition of protein production.

RNAi is highly effective due to a predictable design of active sequences of siRNA and to the targeting of mRNA. When siRNA duplexes are introduced by transfection with a vector, transfection reagent, and delivered into the cytoplasm, RNAi has been shown to effectively silence both exogenous and endogenous genes in a variety of mammalian cells, including cell lines (Elbashir et al, 2001) as well as primary cells.

RNAi is a powerful tool for human therapy which would dramatically drop developments of new therapy approaches for severe diseases such as cancer or viral infections. For exploiting the vast potential of RNAi, the generation of RNAi transfection vectors and strategies developed for efficient delivery to cells and tissues of diseased organisms is required.

The success of RNAi depends on both siRNA (design and chemistry) and vector/carrier for cell delivery. As compared to antisense or ribozyme technology, the secondary structure of the target mRNA (may not be) is not a strong limiting factor for silencing with siRNA. Many sequences of siRNA may be effective for one targeted mRNA. The stability of siRNA duplexes and the amount of siRNA delivered to cells is the most limiting factors for silencing rather the target accessibility by the chosen sequence. Two approaches are proposed for introducing siRNA into cells: the delivery by transfection of synthetic siRNA duplexes into the cytoplasm of cells and the delivery of siRNAs expressed in situ from a plasmid (or DNA cassettes) preliminary introduced by gene transfer into the nucleus.

RNAi in mammalian cells depends upon efficient intracellular delivery of either siRNAs or DNA vector expressing si/shRNAs or microRNA-adapted short hairpin RNA (shRNAmir) (Sui et al., 2002; Yu et al., 2002; Miyagishi & Taira, 2002; Silva et al., 2005; Brummelkamp et al., 2002). Expression of shRNAs (short hairpin RNAs) or siRNAs in mammalian cells can be achieved via transcription from either Pol II or Pol III (U6 or H1) promoters. DNA vectors are based on plasmid and viral vector systems that express double-stranded short hairpin RNAs (shRNAs) that are subsequently processed to siRNAs by the cellular machinery. Recent developments of shRNA systems allow tissue-specific and inducible knockdown of genes. Intracellular delivery of such DNA vectors expressing active RNAs for RNA interference can be achieved by using recombinant viruses or non-viral delivery systems.

Coming to the gene delivery technology, potent viral or non-viral vectors are useful for introducing siRNA duplexes in cells. For mammalian cells in culture, viral vectors appear a potent tool for the production of an intracellular pool of siRNAs expressed from delivered plasmid DNA because of their transduction efficiency and facility to deliver DNA into the nucleus. However, recombinant viral delivery systems still suffer from their immunogenicity and potential risk in clinical situations. In contrast, the transfection of nucleic acids (plasmids or synthetic siRNAs) with synthetic systems is a versatile method showing flexibility and absence of immunogenicity. The transfection of synthetic siRNA duplexes (chemically or enzymatically produced) with non-viral vectors is the best technology to delivery mainly short double-stranded RNA into the cytoplasm. The most efficient non-viral vectors for siRNA delivery are based on cationic lipids-mediated transfection coming initially from the field of gene delivery or newly developed for the specific RNAi application. Cationic lipids formulations compact nucleic acids (plasmid, oligonucleotides, siRNA duplexes) into positively charged particles capable of interacting with anionic proteoglycans at the cell surface and entering cells by endocytosis. After trafficking towards intracellular vesicles of endocytosis, mainly endosomes, the cationic lipids have the property to destabilize the membrane of these intracellular compartments by lipids exchange/diffusion allowing a nucleic acids ‘decomplexation’ and release into cytoplasm (Xu and Szoka, 1996). In addition, a secondary mechanism called proton sponge activity may be associated with some lipids inducing endosomes swelling and rupture which release thus nucleic acids in the cytoplasm. As RNAi mechanism occurs in the cytoplasm, vectors based on formulation of cationic lipids are efficient vehicles to deliver synthetic siRNA duplexes into cells. For siRNA expressed in situ from plasmid, non-viral vectors, based on cationic lipids formulations or cationic polymers, destabilizing endosomal compartments are suitable.

In contrast to their ability to transfect efficiently a gene (long double stranded DNA) into cells, cationic polymers are poorly efficient for the delivery of short nucleic acid. Cationic polymers are shown to be less efficient for siRNA delivery than cationic lipid-based systems. Cationic polymers are able to mediate RNA interference in vitro with concentrations of siRNA around 100 to 200 nM. Selectivity of RNA interference at such concentrations is a limit of their use. In addition, the high amount of siRNA used is correlated with a high amount of polymer which induces cytotoxic effects. To date, cationic polymers such as branched or linear polyethylenimines, poly-histidyl polymers, chitosan, poly(amino ester glycol urethane), amino cyclodextrin derivatives were used in vitro but without relevant efficiency compared to cationic lipids.

An objective of the inventors was to increase the potency of cationic polymers, this major class of non-viral delivery vectors, for in vitro siRNA transfection. Cationic polymers are able to interact via electrostatic interactions between the phosphates of siRNA and the amino groups of polymer. However, according to the structure of siRNA, polymers are unable to condense such small double helix comprising only two turns (about 20 nucleotides per strand). Even complexation occurs between siRNA and cationic polymer leading to siRNA sticking along the polyamine backbone, cationic polymers lack cooperative interactions to induce a condensation into particles or micro-aggregates of molecules complexed.

In addition to electrostatic interactions between positive and negative charges bearing by the polyamine and the small double stranded oligonucleotide, hydrophobic stacking with the nucleic bases and hydrogen bond forming interactions is a way we propose to increase interactions between polyamine and siRNA. Taking together, electrostatic and hydrophobic interactions as well as hydrogen bonds provide enough energy leading to stable complexation and condensation of siRNAs.

Aromatic amino acids (AAA) are responsible of the hydrophobic characteristics in protein and are involved in interactions between protein-protein and protein-ligand via hydrophobic interactions. AAAs are also able to interact with nucleic acid by stacking with the nucleic bases (guanidine, adenosine, thymine or cytosine).

The invention relates to a new concept of hydrophobic polyamines which comprised a polyamine backbone highly modified with aromatic amino acids. This kind of polymer offers the possibility to interact with small nucleic acids, like siRNAs, via electrostatic interactions, hydrophobic stackings and hydrogen bonds. As a barrier of energy to overcome, addition by chemical grafting of AAA to polyamine will be able to provide the sufficient energy to induce cooperative interactions ending in condensation. Consequence is the stabilization of the complex generated by hydrophobic interaction under stable particles or aggregates.

The present invention describes a new class of non viral transfection agents, belonging to the cationic polymers group, which are particularly adapted for the transfection of small sized oligonucleotides. Especially the physical properties of small oligonucleotides prompted the inventors to design a new class of transfection agents based on hydrophobic and cationic polymers.

The inventors have found that transfection agents of high efficiency could be obtained by combining an oligonucleotide of interest with hydrophobic and cationic polymers forming stable complexes of transfection.

Advantageously, said agents are also useful for co-transfection of siRNA with plasmid DNA that can promote in situ expression of small RNAs mediating RNA interference.

It is then an object of the invention to provide new compositions useful as transfection agents for siRNA and optionally DNA vector expressing active RNAs for RNAi.

The invention also relates to a method of transfection of cells in vitro.

The compositions of the invention useful as transfection agents comprise polyamines modified by aromatic amino acids and small double-strand or single-strand RNA.

Advantageously, the polyamines comprise branched or linear polyethylenimine, polyallylamine, dendrimers, polyaminoester, polylysine, polyhistidine, polyarginine, polyornithine or chitosan.

The polyamines are more particularly selected in the group comprising linear polyethylenimine (LPEI), polyallylamine (PAA) and polylysine (PLL).

Preferably, the molecular weight of said polyamines is above 400 Da.

Useful polyamines are selected in the group comprising linear polyethylenimine of 2 KD to 220 KD, polyallylamine of 10 KD to 70 KD and polylysine of 1 KD to 300 KD.

The aromatic amino acids used to modify the polyamines are selected in the group comprising tyrosine, tryptophan and phenylalanine or the derivatives thereof.

Preferably, the aromatic amino acids are tryptophan and/or tyrosine.

In the above defined compositions, the RNA is normal or modified, the modification groups being for example 2′-Fluo, 2′-Methoxy, phosphorothioate, LNA or morpholino.

The above defined RNA is double stranded or single stranded antisens siRNA or mixtures of single stranded sens/antisens siRNA.

Advantageously, the siRNA has 15-30 mers.

A preferred composition comprises the polyamines modified by aromatic amino acids such as above defined and double stranded or single stranded siRNA in an isotonic medium, for example NaCl, glucose, a buffer.

The concentration of siRNA may vary from picomolar to micromolar.

Advantageously, the above defined compositions comprise one or several additives such as PEG, PVA, saccharide, polysaccharide, peptide, protein, vitamins.

According to an embodiment of the invention, the above defined composition further comprises plasmid DNA expressing active RNAs for RNAi or encoding a transgene.

Said plasmid particularly expresses siRNA, shRNA or mino-RNA-adapted short hairpin RNA.

Said siRNA, respectively, can comprise groups stabilized against degradation with suitable groups, selected in the group comprising purine nucleotides, pyrimidine nucleotides substituted by modified analogs such as deoxynucleotides, and/or modified nucleotide analogs such as sugar- or backbone modified ribonucleotides or deoxyribonucleotides. The oligonucleotides sequences can contain deoxyribonucleotides, ribonucleotides or nucleotide analogs (Verma and Eckstein, 1998), such as methylphosphonate, morpholino phosphorodiamidate, phosphorothioate, PNA, LNA, 2′ alkyl nucleotide analogs.

It is another object to provide a process for the synthesis of said compositions.

The method for synthesizing the polyamines modified by aromatic amino acids of the above defined compositions comprise the use of super-ester of aromatic amino acids activated by Dimethoxytriazine-N-methylmorpholium (DMTMM) in the presence of the polyamines.

Advantageously, synthesis of the polyamines is carried out in a basic buffer such as a borate buffer 200 mM, pH=7.5-9 or in an aqueous medium in the presence of a base or a water/alcohol mixture.

The percentage of modification of polyamines by aromatic amino acids in said composition more particularly varies from 0.01% to 100%, particularly of 15% to 50%.

The invention also relates to a method for in vitro, ex-vivo and in vivo transferring siRNA or siRNA and plasmid DNA, comprising using a composition such as above defined.

The in vitro transfer of siRNA is advantageously carried out in a medium culture containing adherent cells or cells in suspension.

The medium is a normal medium or synthetic medium.

The invention also provides compositions for use as pharmaceutical compositions for inducing a regulating effect on the expression of one or more target proteins responsible or involved in genetic hereditary diseases or complex genetic diseases.

It is an object of the invention to provide new compositions useful as DNA vector transfection agents that can promote in situ expression of small RNAs mediating RNA interference.

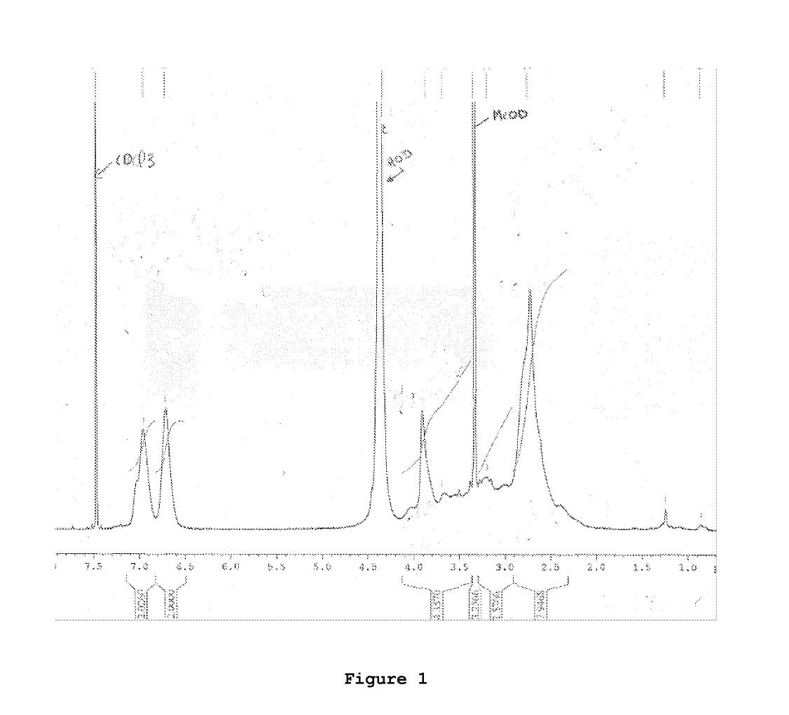

Other characteristics and advantages of the invention are given in the following examples wherein it is referred to FIGS. 1-8, which respectively relate to:

FIG. 1: ¹H-NMR analysis of L-PEI-Tyr conjugate in D₂O.

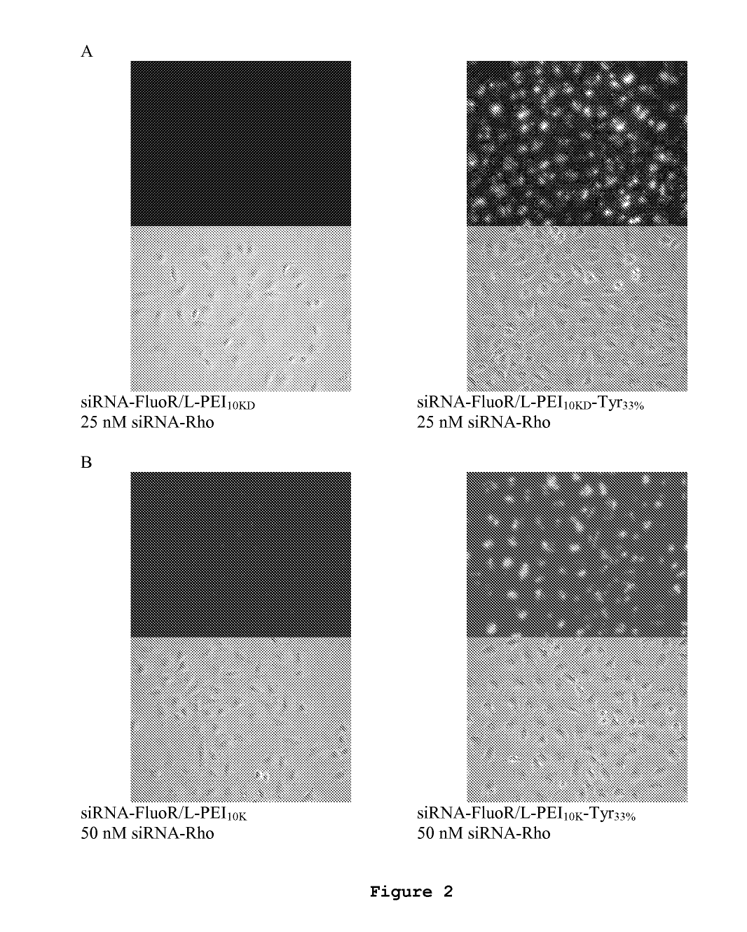

FIGS. 2A and 2B: siRNA delivery in A549 cells.

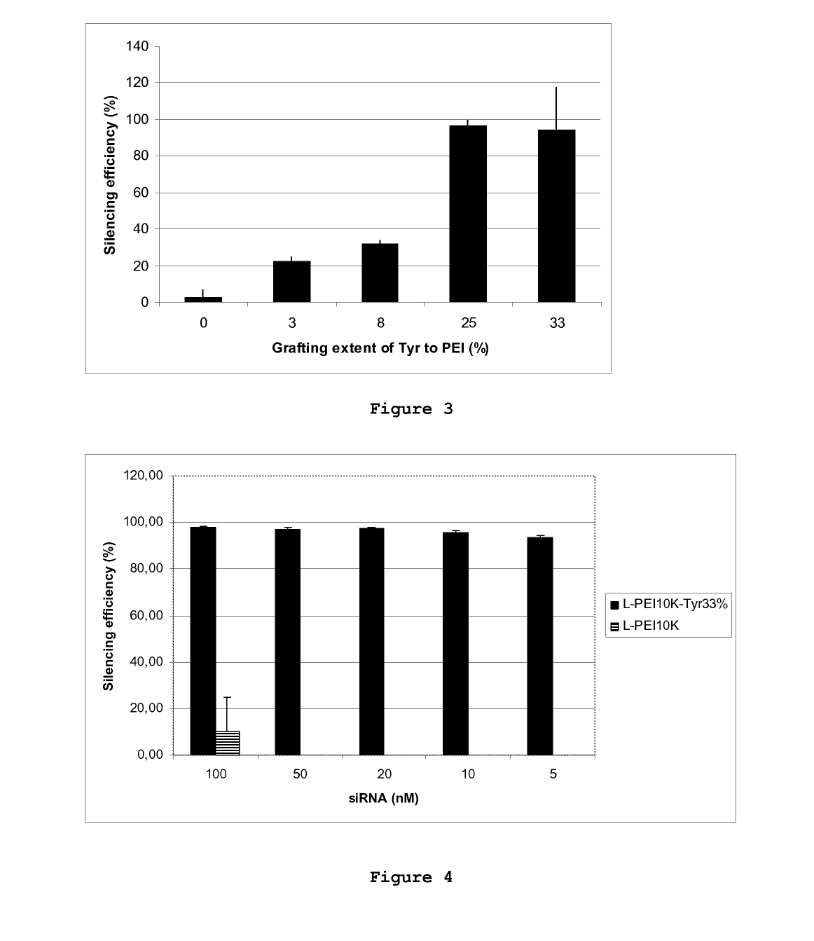

FIG. 3: RNA interference efficiency of luciferase gene (pGL3) stably expressed by A549-GL3Luc cells by GL3Luc siRNA transfected with the L-PEI_{10K} modified with different extents of tyrosine residue.

FIG. 4: comparative silencing efficiency of luciferase gene (pGL3) stably expressed by A549-GL3Luc cells by GL3Luc siRNA transfected with the L-PEI_{10K} or L-PEI_{10K}Tyr_{33%} conjugate.

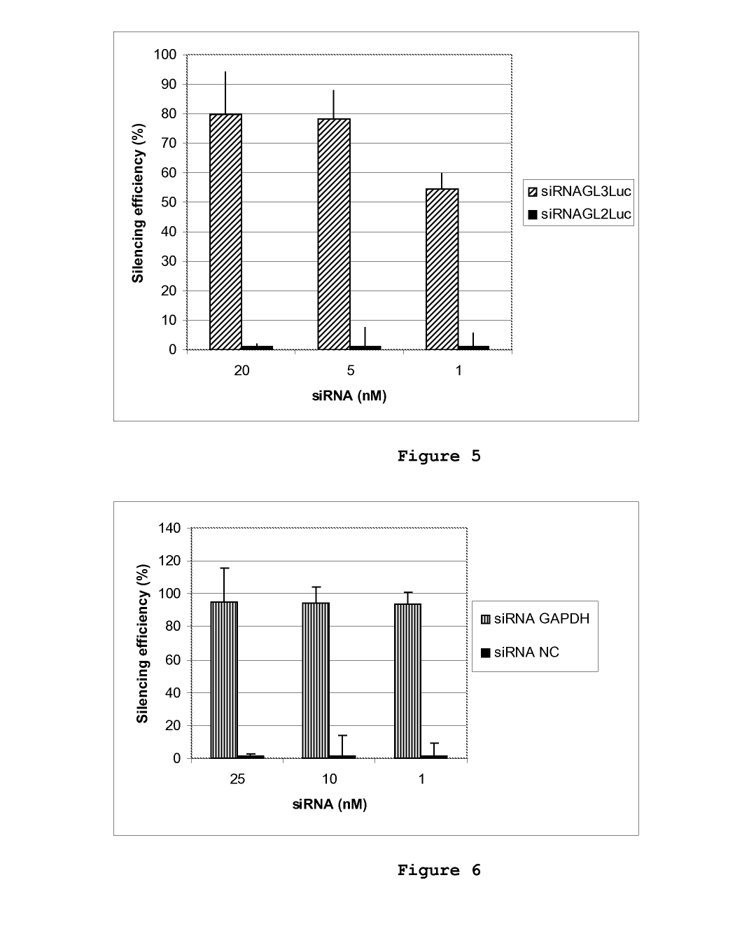

FIG. 5: Selective RNA interference of luciferase gene (pGL3) stably expressed by A549-GL3Luc cells by GL3Luc siRNA transfected with the L-PEI_{10K}-Tyr_{33%}.

FIG. 6: Efficient and selective GAPDH gene silencing in HeLa cells lines after transfection of siRNA complexed with l-PEI_{10K}-Tyr_{33%}.

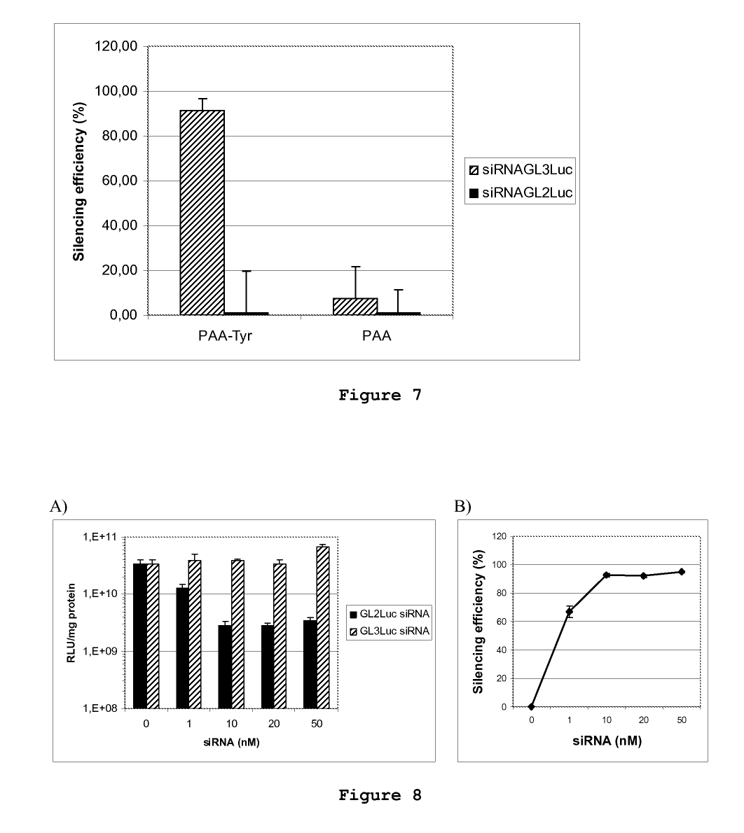

FIG. 7: Selective RNA interference of luciferase gene (pGL3) stably expressed by A549-GL3Luc cells by GL3Luc siRNA transfected with the PAA_{17K}-Tyr₄₀%.

FIGS. 8A and 8B: Selective RNA interference of luciferase gene (GL2Luc) expressed by HeLa cells after co-transfection of GL2Luc siRNA and pCMVLuc plasmid (pGL2Luc) with the PEI_{10K}-Tyr₁₉%.

### MATERIAL AND METHODS

### Chemicals and Oligonucleotides

Oligonucleotides were chemically synthesised and PAGE purified by Eurogentec (Belgium). Oligonucleotides were annealed in 1× Annealing buffer (50 mM K-Acetate, 50 mM Mg-Acetate) (Eurogentec) for 2 min. at 95° C., followed by 2-4 hours incubation at room temperature. GAPDH SMART pool® reagent was from Dharmacon.

SiRNA duplexes used correspond to sequences SEQ ID N° 1 and SEQ ID N° 2; SEQ ID N° 3 and SEQ ID N° 4; SEQ ID N° 5 and SEQ N° 6; SEQ ID N° 7 and SEQ ID N° 8

| | | |
|---|---|---|
| GL3Luc siRNA duplex | SEQ ID N^{o} 1 | 5′-CUUACGCUGAGUACUUCGA(dT)₂-3′ |
| | SEQ ID N^{o} 2 | 3′-(dT)₂GAAUGCGACUCAUGAAGCU-5′ |
| GL2Luc siRNA duplex | SEQ ID N^{o} 3 | 5′-CGUACGCGGAAUACUUCGA(dT)₂-3′ |
| | SEQ ID N^{o} 4 | 3′-(dT)₂GCAUGCGCCUUAUGAAGCU-5′ |
| siRNA TNF-alpha | SEQ ID N^{o} 5 | 5′-GCACCACUAGUUGGUUGUC(dT)₂-3′ |
| Rhodamine duplex | SEQ ID N^{o} 6 | 3′-dT)₂CGUGGUGAUCAACCAACAG-5′ |
| Lamin A/C siRNA duplex | SEQ ID N^{o} 7 | 5′-CUGGACUUCCAGAAGAACAdTdT-3′ |
| | SEQ ID N^{o} 8 | 3′-dTdTGACCUGAAGGUCUUCUUGU-5′ |

All reagents for chemistry and starting material were purchased from Sigma-Aldrich (France) and were used without prior purification. Solvents were ordered from SDS-Carlo Erba (France). Diethylether was dried and distilled over sodium benzophenone.

¹H NMR spectra were recorded with a Bruker AF-400 spectrometer at 25° C. in CDCl₃, D₂O or CD₃OD and proton chemical shifts are reported downfield from TMS. NMR multiplicities are abbreviated as s=singlet, d=doublet, br=broad, m=multiplet, t=triplet

Synthesis of linear polyethylenimine (L-PEI)

LPEI is obtained from the intermediate poly-2-ethyl-2-oxazoline generated after the living cationic ring opening polymerization of 2-ethyl-2-oxazoline monomer.

Synthesis of poly-2-ethyl-2-oxazoline: 0.4 moles 2-ethyl-2-oxazoline monomer were dissolved in 40 ml acetonitrile then 0.4/X moles of methyl p-toluenesulfonate are added under argon atmosphere. The reaction was heated at 80° C. for 24-48 hours. The reaction was quenched with saturated aqueous Na₂CO₃ and heated for 24 hours. After slow cooling at room temperature, 10 ml of methanol and ether were added until precipitation.

The precipitate was filtered and washed with ether. Poly-2-ethyl-2-oxazoline was obtained with 80-90% yield.

¹H-NMR analysis, 400 MHz, in CDCl₃: 1-1.06 ppm (s, 3H, CH₃CH₂CONCH₂CH₂); 2.2-2.3 ppm (m, 2H, CH₃CH₂CONCH₂CH₂); 3-3.4 ppm (s, 4H, CH₃CH₂CONCH₂CH₂).

Synthesis of L-PEI: 0.35 moles of poly-2-ethyl-2-oxazoline were dissolved in 100 mL of water, then 200 mL of hydrochloric acid 37% were added and the mixture is heated at 120° C. After 24 hours, the reaction mixture was evaporated and then water was added before lyophilisation. The yield was 90%.

Analysis by ¹H-NMR, 400 MHz in D₂O: single peak at 3.4 ppm

| 0.4/X moles of | X = number | Mw of poly-2- | |
|---|---|---|---|
| methyl p- | of | ethyl-2- | Mw of |
| toluenesulfonate | monomer | oxazoline | polyethylenimine |
| 8 mmoles | 50 | 5 KDa | 2 KDa |
| 1.6 mmoles | 250 | 25 KDa | 10 KDa |
| 0.8 mmoles | 500 | 47 KDa | 20 KDa |

Synthesis of polyethylenimine-aromatic α-amino acid conjugate

Synthesis of N,O-Boc-tyrosine

Five g of L-tyrosine (Sigma) are dissolved in 125 mL of Na₂CO₃ (0.1 mg/mL) and 50 mL of THF. Then, 17.2 g of Boc₂O dissolved in 75 mL de THF are added onto the tyrosine solution and the mixture was stirred for 3 days at room temperature. Water (40 ml) was added and N,O-Boc-tyrosine was extracted with ether. The acqeuous phase was acidified with HCl and 2 novel extractions with ethyl acetate were performed. After evaporation, the raw product is purified by chromatography on silica gel (in 5% MeOH/CH₂Cl₂). 5.2 g of N,O—BOC-Tyrosine were obtained.

Analysis by ¹H-NMR, 400 MHz, in CDCl₃: 7.2 ppm (d, 2H, aromatic H), 7.1 ppm (d, 2H, aromatic H), 4 ppm (m, 1H, BocO—Ar—CH₂—CH(NHBOC)—COOH), 3-2.99 ppm (dd, 1H, BocO—Ar—CH₂—CH(NHBOC)—COOH), 2.84-2.78 ppm (dd, 1H, BocO—CH₂—CH(NHBOC)—COOH), 1.48 ppm (S, 9H, BocO—Ar—CH₂—CH(NHBOC), 1.32 ppm (S, 9H, BocO—Ar—CH₂—CH(NHBOC)—COOH).

### Synthesis of N-Boc-tryptophane

Synthesis of N-Boc-tryptophane was realized with the same protocol used for the synthesis of N,O-Boc-tyrosine, starting with 2 g of tryptophane and 6.5 g of Boc₂O, to give 2.79 g of white solid

Analysis by ¹H-NMR, 400 MHz, in CDCl₃: 8.1 ppm (s, 1H, —COOH) 7.6 ppm (d, H, aromatic H), 7.38 ppm (d, 1H, aromatic H), 7.2 ppm (m, 1H, aromatic H), 7.16 (m, 1H, aromatic H), 7.01 ppm (m, 1H, aromatic H), 5.09 ppm (d, 1H, NHBOC), 4.68 ppm (br, 1H, BocO—Ar—CH₂—CH(NHBOC)—COOH), 3.35 ppm (m br, 2H, BocO—Ar—CH₂—CH(NHBOC)—COOH), 1.45 ppm (S, 9H, BocO—Ar—CH₂—CH(NHBOC).

### Synthesis of Polyethylenimine-Tyrosine Conjugate (L-PEI-Tyr)

### Synthesis of L-PEI_{10KD}-Tyr_{33%}

Protocol 1: 100 mg of L-PEI_{10K}.HCl (1.26 mmoles) were dissolved in 5 mL of 200 mM borate buffer, pH=8.2. Then, pH was adjusted to 8 with 10N NaOH. 240 mg of N,O-Boc-Tyrosine (0.632 mmoles) dissolved in 15 mL of THF were added and the mixture was stirred for 10 minutes. DMTMM (500 mg) sont added into the mixture and stirred for 48 hours. After evaporation, the solid was washed with water. After drying, 239 mg of yellow solid were obtained (L-PEI_{10K}-TyrBoc₂). 185 mg de LPEI-TyrBoc₂ were dissolved in 5 mL of trifluoroacetic (TFA). After 3 hours, the reaction mixture was evaporated and dialysed in water.

After lyophilization, 111 mg of white solid was obtained (L-PEI_{10K}-Tyr)

Protocol 2: To 200 mg de L-PEI_{10K}.HCl (2.53 mmoles) in 4 mL of water, 0.56 mL de N-méthylmorpholine and 483 mg de N,O-Boc-Tyrosine (0.5 equivalent, 1.26 mmoles) in 12 mL of methanol were added. The reaction mixture were stirred for 30 minutes and 700 mg de DMTMM were added. After 48 hours, the reaction mixture was evaporated and the solid was dissolved in 8 mL de TFA. After 3 hours, the reaction mixture was evaporated and then dialysed in water one day and in HCl 2N two days. After lyophilization, 260 mg of white solid was obtained.

¹H NMR, 400 MHz, CDCl₃ (FIG. 1): 6.9 ppm (s br, 2H, H aromatic), 6.7 ppm (s br, 2H, H aromatic), 3.8 ppm (s, 4H, TyrCONCH₂CH₂), 3.68 ppm (br, 1H, ArCH₂CH(NH₂)COPEI), 3.1 ppm (br, 2H, ArCH₂CH(NH₂)COPEI), 2.74 ppm (s br, 4H, NHCH₂CH₂).

### Synthesis of Polyethylenimine-Tryptophan Conjugate (L-PEI-Trp)

### Synthesis of L-PEI_{10KD}-Trp_{33%}

Synthesis of L-PEI-Trp was realized with the same protocol used for the synthesis of L-PEI-Tyr, starting with 0.1 g of LPEI and 0.193 g of N-Boc-tryptophane.

¹H NMR, 400 MHz, D₂OD: 7.4-7.1 ppm (m br, 5H, H aromatic), 3.91 ppm (m br, 3H, ArCH₂CH(NH₂)COPEI), 3.47 ppm (s br, 4H, ArCONCH₂CH₂ and HNCH₂CH₂).

### Cell Culture

HeLa (human cervix epithelial adenocarcinoma, CCl-2) cells were grown in MEM (Eurobio) supplemented with 2 mM glutamax (Eurobio), Earle's BSS (Eurobio), 1.5 g/L sodium bicarbonate (Eurobio), 0.1 mM non-essential amino acids (Eurobio), 1.0 mM sodium pyruvate (Eurobio), 100 units/ml penicillin (Eurobio), 100 μg/ml streptomycin (Eurobio), and 10% of FBS (Perbio).

A549 (human lung carcinoma, ATCC N° CCL-185) cells stably expressing the GL3 luciferase (Photinus pyralis luciferase under the control of SV40 elements) were obtained after stable transfection of pGL3Luc plasmid (Clontech). A549-GL3Luc cells were grown in RPMI-1640 and supplemented with 10% fetal bovine serum, 2 mM glutamax, 100 units/ml penicillin, 100 μg/ml streptomycin and 0.8 μg/ml G418 (Promega). All the cells were maintained at 37° C. in a 5% CO₂ humidified atmosphere.

### Transfection Experiments

One day before transfection, 2.5×10⁴ cells were seeded in 24-well tissue culture plate in 1 ml fresh complete medium containing 10% FBS. Before transfection, complexes of siRNA/polymer were prepared. The desired amount of siRNAs was diluted in 50 μl of 50 mM phosphate buffer, pH 6 or 8. Then, the desired volume of polymer solution (7.5 mM nitrogen) was added into the siRNA solution. The resulting solution was mixed with a Vortex for 10 seconds and left for 10-15 minutes at room temperature. Before adding the transfection solution onto the cells, the complete medium was removed and replaced by 0.55 ml of fresh complete medium containing 10% FBS. Then, 50 μl of complexes solution were added per well and the plates were incubated at 37° C.

### Co-Transfection Experiments

One day before transfection, 5×10⁴ cells were seeded in 24-well tissue culture plate in 1 ml fresh complete medium containing 10% FBS. Before transfection, complexes with polymer, plasmid and siRNA were prepared. One hundred ng of pCMVLuc (GL2Luc duplex sequence) desired amount of siRNAs were diluted in 50 μl of 50 mM phosphate buffer, pH 7. Then, 2 μl of l-PEI_{10K}-Tyr_{19%} solution (7.5 mM nitrogen) were added into the plasmid and siRNA solution. The resulting solution was mixed with a Vortex for 10 seconds and left for 10-15 minutes at room temperature. Before adding the transfection solution onto the cells, the complete medium was removed and replaced by 0.55 ml of fresh complete medium containing 10% FBS. Then, 50 μl of complexes solution were added per well and the plates were incubated at 37° C. Luciferase gene expression was measured after 24 h incubation period. Experiments were made in triplicates and the luciferase activity was expressed as Relative Light Unit (RLU) normalized by the content of protein in the cell lysates (mg of protein). Then, the silencing efficiency was calculated from the ratio of luciferase activities from GL2Luc siRNA- and GL3Luc siRNA-transfected cells.

### DNA Vector Based RNAi Technology Transfection Protocol

One day before transfection, 5×10⁴ cells were seeded in 24-well tissue culture plate in 1 ml fresh complete medium containing 10% FBS. Before transfection, complexes with polymer and DNA vector expressing short RNA mediating RNAi (siRNA, shRNA, microRNA-adapted short hairpin RNA), were prepared. Plasmid RNAi vector (1 μg) was diluted in 50 μl of 50 mM phosphate buffer, pH 7. Then, 2-4 μl of l-PEI_{10K}-Tyr_{19%} solution (7.5 mM nitrogen) were added into the Plasmid RNAi vector solution. The resulting solution was mixed with a Vortex for 10 seconds and left for 10-15 minutes at room temperature. Before adding the transfection solution onto the cells, the complete medium was removed and replaced by 0.55 ml of fresh complete medium containing 10% FBS. Then, 1 to 50 μl of complexes solution were added per well and the plates were incubated at 37° C. After one day of incubation, 0.4 ml of complete fresh medium was added. The level of the targeted gene expression (mRNA level) or inhibition of the protein production (protein level) was determined 24 to many days later. As a control, plasmid RNAi vector expressing a non-specific active RNA (containing a mismatch sequence) against the targeted gene expression was used.

### Luciferase and Protein Assay

Luciferase gene expression was measured using a commercial kit (Promega, France). After removing the complete medium, three washings with 1 ml of PBS solution were made. Then, 100 μl of 1× lysis buffer were added per well, and the plate was incubated at room temperature for 30 minutes. The lysates were collected and centrifuged at 14,000 g for 5 minutes. The luciferase assay was assessed with 2.5 μl of lysate after injection of 100 μl of luciferin solution. The luminescence (RLU) was monitored with an integration over 5 seconds with a luminometer (LB960, Berthold, France). Results are expressed as light units integrated over 10 seconds (RLU), per mg of cell protein using the BCA assay (Pierce, France).

Measurement of mRNA Level

Messager RNA level was determined by the QuantiGene®Branched DNA assay (GenoSpectra) which is performed with whole cell lysates and without target amplification.

After 48 h transfection, HeLa cells were washed with 1 mL PBS 1× (Cambrex) and lysed in 0.6 mL of 1× Genospectra lysis buffer for 30 min. at 50° C. Then, the plate was stored at −80° C. for at least 30 min. The lysates were thawed and 2 to 20 μl of lysate were adding to the capture plate. Ten μl of lysis working reagent (for 48 reactions, the lysis working reagent is prepared by adding 25 μl of CE (capture extender), 25 μl of LE (label extender) and 25 μl of BL (blocking probe) and 425 μl of 3× lysis mixture, all compounds are from Genospectra) were added to the plate and the volume was completed to 100 μl with 1× lysis mixture. The plate was covered with a lid and incubated for 16 h at 50° C. The plate was washed 3 times with 300 μl of 1× wash buffer (Genospectra), and 100 μl of Amplifier working solution (0.116 μl of amplifier diluted in 116 μl Amplifier diluent, all from Genospectra) were added to each well. The plate was incubated for 1 hour at 50° C. After 3 times 1× wash buffer washing, 100 μl of Label Probe Working Reagent (0.116 μl of label probe diluted in 116 μl Amplifier diluent, all from Genospectra) were added to each well and incubated for 1 hour at 50° C. The plate was then washed 3 times with 1× wash buffer and 100 μl of Substrate Working Reagent (0.348 μl of 10% Lithium Lauryl sulphate in 116 μl of Substrate, all from Genospectra) was added to each well. After 30 minutes incubation, the luminescence was measured in each well with a spectrophotometer (Berthold).

### Fluorescence Microscopy

One day before transfection, 2.5×10⁴ A549 cells were seeded in 24-well tissue culture plate in 1 ml fresh complete medium containing 10% FBS. Before transfection, complexes of siRNA/polymer were prepared. The desired amount of siRNA-Rhodamine (Rho) was diluted in 50 μl of 50 mM phosphate buffer, pH 6 or 8. Then, the desired volume of L-PEI_{10K}-Tyr_{33%} solution (7.5 mM nitrogen) was added into the siRNA solution. The resulting solution was mixed with a Vortex for 10 seconds and left for 10-15 minutes at room temperature. Before adding the transfection solution onto the cells, the complete medium was removed and replaced by 0.55 ml of fresh complete medium containing 10% FBS. Then, 50 μl of complexes solution were added per well and the plates were incubated at 37° C. for 24 or 48 hours.

Before observation, cells were washed with 1 ml of PBS-BSA1% and then observed by fluorescence microscopy (ECLIPSE TE2000-S, Nikon).

### Results

Linear polyethylenimine (L-PEI) having a mean molecular weight of 10 kDa was produced using cationic ring opening polymerization of 2-ethyl-2-oxazoline monomer. Then, L-PEI_{10K} was modified with tyrosine residues at various extents following the protocols 1 or 2 as described in the Material and Methods. All L-PEI-Tyr derivatives were characterized by ¹H-NMR as exemplified in FIG. 1.

SiRNA Delivery into Cells in Culture with L-PEI-Tyr Derivatives.

Comparative siRNA delivery potency into cell in culture with linear polyethylenmine and tyrosine modified linear polyethylenimine (L-PEI_{10K}Tyr_{33%}) was investigated using fluorescent siRNA (rhodamine-labelled siRNA, siRNA-FluoR). Small amounts of siRNA-FluoR (final concentration 25 and 50 nM) were complexed with 2 μl of L-PEI_{10K} or L-PEI_{10K}Tyr_{33%} (both stock solutions at 7.5 mM nitrogen) in 50 μl of 50 mM phosphate buffer, pH 6.0. The resulting transfection was added onto A549 cells cultured in complete culture medium containing 10% FBS. Cells were incubated in 24-well tissue culture plate format, for 24 hours before their observation by fluorescence microscopy. The results are given on FIG. 2 incubation in 1 ml of complete cell culture medium containing 10% FBS and with 25 (A) or 50 nM (B) siRNA-Rhodamine (Rho) complexed with 2 μl of L-PEI10K or L-PEI_{10K}-Tyr_{33%} (7.5 mM nitrogen) in 50 μl of 50 mM phophate buffer pH 6.0. Before observation, cells were washed with 1 ml of PBS-BSA1% and then observed by fluorescence microscopy (ECLIPSE TE2000-S, Nikon), magnification ×200.

Rare punctuate fluorescence within the cytoplasm of cells was observed with L-PEI_{10K}-mediated delivery for the both siRNA concentrations tested. Intense and punctuate fluorescence within all areas of the cytoplasm was observed after siRNA delivery with L-PEI_{10K}Tyr_{33%}. These results show that cellular uptake of siRNA is very efficient using L-PEI_{10K}Tyr_{33%} comparatively to the unmodified polyamine.

As a target model for testing the efficiency of the polymers of the invention to mediate the silencing of endogenous reporter gene, we used the A549 cells stably expressing the GL3 luciferase (Photinus pyralis luciferase under the control of SV40 elements). Cells were transfected in 0.55 ml of complete culture medium containing 10% FBS (in 24-well tissue culture plate format) with GL3Luc siRNA, at 20 nM, complexed with 2 μl of l-PEI_{10K}-Tyr_{x%} (7.5 mM nitrogen) in 50 μl of 50 mM phosphate buffer, pH 6.0. Luciferase gene expression was measured after 48 h incubation period. Experiments were made in triplicates and the luciferase activity was expressed as Relative Light Unit (RLU) normalized by the content of protein in the cell lysates (mg of protein). Then, the silencing efficiency was calculated from the non-transfected cells.

A well defined (validated by Elbashir et al., 2001) and conventional siRNA (siRNAGL3Luc), chemically produced, and sequence-specific GL3Luc siRNA composed of a short dsRNA of 19 nucleotides matching the GL3Luc mRNA and comprising 3′-overhangs of 2 deoxyribonucleotides (dT) was used for the transfection experiments. SiRNA was complexed with unmodified L-PEI_{10K} or modified with tyrosine residue at different extents (3, 8, 25, or 33% of nitrogen modification per polymer) in 50 mM phosphate buffer, pH 6.0. The resulting solution of transfection complexes was added on the cells growing in medium containing serum and cells were finally exposed to siRNA concentration of 20 nM. The results are given on FIG. 3. The silencing efficiency was determined 48 h post-transfection by measuring the luciferase activity with a standard luminescence assay normalized by the protein content of cell lysates. The luciferase activity (expressed as RLU/mg of protein) was not significantly inhibited (<2%) when the transfection was performed with the unmodified polyamine. When polyamine-tyrosine conjugates were used, the silencing efficiency increased as a function of the grafting extent of tyrosine to polyamine to reach a plateau for 25-33% of modification with 90-95% inhibition of luciferase activity.

Comparative Silencing Efficiency of Luciferase Gene (pGL3) Stably Expressed by A549-GL3Luc Cells by GL3Luc siRNA Transfected with the L-PEI_{10K} or L-PEI_{10K}Tyr_{33%} conjugate.

Selective silencing after polymer-mediated siRNA delivery was assessed with A549 cells stably expressing the GL3 luciferase (Photinus pyralis luciferase under the control of SV40 elements). The cells were transfected in 0.55 ml of complete culture medium containing 10% FBS (in 24-well tissue culture plate format) with GL3Luc siRNA, 1 to 100 nM, complexed with 2 μl of l-PEI_{10K} or l-PEI_{10K}-Tyr_{33%} conjugate (7.5 mM nitrogen) in 50 μl of 50 mM phosphate buffer, pH 6.0. Luciferase gene expression was measured after 48 h incubation period. Experiments were made in triplicates and the luciferase activity was expressed as Relative Light Unit (RLU) normalized by the content of protein in the cell lysates (mg of protein). Then, the silencing efficiency was calculated from the non-transfected cells.

Specific siRNAGL3Luc was complexed with L-PEI_{10K}Tyr_{33%} in 50 mM phosphate buffer, pH 6.0. Cells were transfected with 5 to 100 nM siRNA. The luciferase activity, determined 48 hours post-transfection, was inhibited up to 98% when the transfection was performed with 5 to 100 nM siRNA. The results are given on FIG. 4. As control polymer, unmodified L-PEI₁₀ₖ was shown to inhibit in the same conditions the luciferase activity by 10% at 100 nM. However, luciferase activity was not inhibited using lower siRNA concentration from 5 to 50 nM when the transfection was performed with this L-PEI₁₀ₖ.

Specific Gene Silencing Using siRNA/L-PEI₁₀ₖ-Tyr Complex.

Selectivity of luciferase silencing was tested with the non specific siRNA targeting the GL2Luc gene (siRNA validated by Elbashir et al. 2001) in the same conditions of transfection with L-PEI_{10K}Tyr_{33%}. A549-GL3Luc cells, stably expressing the luciferase gene, were transfected in 0.55 ml of complete culture medium containing 10% FBS (in 24-well tissue culture plate format) with GL3Luc siRNA, 1 to 20 nM, complexed with 2 μl of l-PEI_{10K}-Tyr_{33%} (7.5 mM nitrogen) in 50 μl of 50 mM phosphate buffer, pH 8.0. Luciferase gene expression was measured after 48 h incubation period. Experiments were made in triplicates and the luciferase activity was expressed as Relative Light Unit (RLU) normalized by the content of protein in the cell lysates (mg of protein). Then, the silencing efficiency was calculated from the non-transfected cells. The results are given on FIG. 5.

The absence of effect on the luciferase activity when cells were transfected with this unrelated sequence, the GL2Luc siRNA, in the same conditions, confirmed a sequence-specific RNA interference.

Efficient Endogenous Gene Silencing Transfection of siRNA Complexed with l-PEI_{10K}-Tyr_{33%}.

HeLa cells were transfected with GAPDH siRNA (1 to 25 nM) complexed with 2 μl of l-PEI_{10K}-Tyr_{33%} (7.5 mM nitrogen) in 50 μl of 50 mM phosphate buffer, pH 8.0. GAPDH mRNA level was measured by branched DNA assay after 48 h incubation period and was inhibited by more than 90% using siRNA concentration from 1 to 25 nM. The results are given on FIG. 6. As unspecific control, siRNA matching an unrelated sequence (lamin A/C) was transfected in the same conditions. Unspecific control showed no inhibition effect on the GAPDH mRNA level.

### Cationic Polymers Modified by Hydrophobic Alpha Amino Acids or Derivatives Mediate Efficient Gene Silencing.

The gene silencing improvement following siRNA delivery into cells in culture with polymer modified with tyrosine residues was also exemplified using the polyallylamine (PAA) having a MW of 17 kDa. PAA was grafted with tyrosine residues with modification extent of nitrogen of 40%. Transfection complexes were prepared with siRNAGL3Luc and 1 μl of PAA_{17K}-Tyr_{40%} in 50 μl of 50 mM phosphate buffer, pH 6.0. A549-GL3Luc cells were transfected in 0.55 ml of complete culture medium containing 10% FBS and with GAPDH siRNA (1 to 25 nM) complexed with 2 μl of l-PEI_{10K}-Tyr_{33%} (7.5 mM nitrogen) in 50 μl of 50 mM phosphate buffer, pH 8.0. GAPDH mRNA level was measured by branched DNA assay after 48 h incubation period. As unspecific control, siRNA matching an unrelated sequence (lamin A/C) was transfected in the same conditions. Experiments were made in triplicates and the GAPDH silencing efficiency was calculated from the endogenously GAPDH level of non-transfected cells. The results are given on FIG. 7. PAA_{17K}-Tyr_{40%} provided a silencing of 90% whereas unmodified PAA showed a low and not significant silencing around 10%. In addition, the silencing obtained with PAA_{17K}-Tyr_{40%} was confirmed to be selective because siRNAGL2Luc totally failed to silence the luciferase gene.

Many polymers, including linear or branched polyethylenimine, polyallylamine, or poly-L-Lysine were chemically modified with different hydrophobic alpha amino acids or derivatives such as tyrosine, tryptophane or 3,4-dihydroxy-L-phenylalanine (DOPA) as phenylalanine derivative. Silencing efficiency of these polymer conjugates was tested after transfection of A549 cells stably expressing the GL3 luciferase, using 5 or 20 nM siRNA The results are given in Table 1 hereinafter.

TABLE 1
Silencing efficiency of conjugate samples of polyamines modified by aromatic
α-amino acid residues with different molecular weight and modification extents
(L-PEI: linear polyethylenimine, PAA: polyallylamine, PLK, Poly-L-Lysine).

| | MW kDa | | MW kDa | Silencing | Silencing |
|---|---|---|---|---|---|
| Polymers | (polyamine) | Grafting (%) | (conjugate) | (%) at 20 nM siRNA | (%) at 5 nM siRNA |
| L-PEI-Tyr | 10 | 25 | 31 | 84 +/− 18 | 86 +/− 1 |
| B-PEI-Tyr | 25 | 25 | 75.6 | 96 +/− 1 | 92 +/− 1 |
| PAA-Tyr | 17 | 40 | 47.4 | 91 +/− 5 | nd |
| PLK-Tyr | 22 | 50 | 35 | 87 +/− 11 | 86 +/− 8 |
| L-PEI-Trp | 10 | 33 | 32.5 | 40 +/− 9 | nd |
| PAA-Trp | 17 | 37 | 49.8 | 87 +/− 10 | nd |
| L-PEI-DOPA | 10 | 23 | 27.9 | nd | 80 +/− 24 |

nd: not determined

The molecular weight of each conjugate is calculated from the mean molecular weight of polyamine and from the percentage of modifications by aromatic α-amino acid residues. Silencing efficiency was determined using A549-GL3Luc cells, stably expressing the luciferase gene. Cells were transfected in 0.55 ml of complete culture medium containing 10% FBS (in 24-well tissue culture plate format) with GL3Luc siRNA, at 5 or 20 nM, complexed with 2 μl of conjugate in 50 μl of 50 mM phosphate buffer, pH 6.0. Luciferase gene expression was measured after 48 h incubation period. Experiments were made in triplicates and the luciferase activity was expressed as Relative Light Unit (RLU) normalized by the content of protein in the cell lysates (mg of protein). Then, the silencing efficiency was calculated from the non-transfected cells.

All the polymer conjugates tested showed high silencing (80% and above) of luciferase gene using low concentrations of siRNA.

High modification extent is required to obtain high gene silencing efficiency. This requirement was also confirmed using the same backbone of polymer but having different MW. The results are given in Table 2 hereinafter

TABLE 2
Silencing efficiency of conjugate samples of linear
polyethylenimine of different molecular weight
modified by 25% with tyrosine residues.

| MW kDa | MW kDa | Silencing (%) at 5 nM |
|---|---|---|
| (polyamine) | (conjugate) | siRNA |
| 2 | 6.04 | 67 |
| 10 | 30.2 | 96 |
| 22 | 66.5 | 95 |

The molecular weight of each conjugate is calculated from the mean molecular weight of polyamine and from the percentage of modifications by tyrosine residues. Silencing efficiency was determined using A549-GL3Luc cells, stably expressing the luciferase gene. Cells were transfected in 0.55 ml of complete culture medium containing 10% FBS (in 24-well tissue culture plate format) with GL3Luc siRNA, at 5 nM, complexed with 2 μl of conjugate in 50 μl of 50 mM phosphate buffer, pH 6.0. Luciferase gene expression was measured after 48 h incubation period. Experiments were made in triplicates and the luciferase activity was expressed as Relative Light Unit (RLU) normalized by the content of protein in the cell lysates (mg of protein). Then, the silencing efficiency was calculated from the non-transfected cells.

As shown by said results linear polyethylenimine of 2, 10 or 22 kDa were modified with the same extent of modification with tyrosine residues (25%). All these polymers were able to silence the luciferase gene after transfection of A549-GL3Luc cells with a low siRNA concentration (5 nM).

When analyzed in more details, the high content of hydrophobic alpha amino acids grafted to polyamine strongly increases the mass (MW) of the polymer conjugate (Tables 1 and 2). Particularly, when the initial mass of the polyamine increases 2-3 fold after coupling of hydrophobic alpha amino acids or derivatives, the modified polymer was shown to be efficient for RNA interference. These modified polymers are rather more hydrophobic than cationic. This analysis confirms that hydrophobic interaction drives the overall behavior of polyamine in presence of siRNA and promotes high gene silencing efficiency.

Efficient Gene Silencing after Co-Transfection of Plasmid and siRNA Complexed with l-PEI_{10K}-Tyr.

HeLa cells (50 000 cells/well) were co-transfected in 0.55 ml of complete culture medium containing 10% FBS (in 24-well tissue culture plate format) with pCMVLuc plasmid (100 ng, GL2Luc sequence) and either specific GL2Luc siRNA or mismatch GL3Luc siRNA (0 to 50 nM), complexed with 2 μl of PEI_{10K}-Tyr_{19%} in 50 μl of 50 mM phosphate buffer, pH 7.0. Luciferase gene expression was measured after 24 h incubation period. The results are given on FIG. 8. Experiments were made in triplicates and the luciferase activity (A) was expressed as Relative Light Unit (RLU) normalized by the content of protein in the cell lysates (mg of protein). Then, the silencing efficiency (B) was calculated from the ratio of luciferase activities from GL2Luc siRNA- and GL3Luc siRNA-transfected cells.

Specific GL2Luc siRNA inhibited the GL2 Luciferase expression by more than 90% using siRNA concentration from 10 to 50 nM (FIG. 8). As unspecific control, siRNA matching an unrelated sequence (GL3Luc sequence) was co-transfected in the same conditions. Unspecific control showed no inhibition effect on the luciferase activity. This experiment confirms that l-PEI_{10K}-Tyr conjugates are able to simultaneously co-deliver a plasmid encoding a transgene and a specific and active siRNA.

### REFERENCES

Brummelkamp, T. R., R. Bernards, and R. Agami. 2002. A system for stable expression of short interfering RNAs in mammalian cells. Science. 296:550-3.
Elbashir, S. M., J. Harborth, W. Lendeckel, A. Yalcin, K. Weber, and T. Tuschl. 2001. Duplexes of 21-nucleotide RNAs mediate RNA interference in cultured mammalian cells. Nature. 411:494-8.
Fire, A., S. Xu, M. K. Montgomery, S. A. Kostas, S. E. Driver, and C. C. Mello. 1998. Potent and specific genetic interference by double-stranded RNA in Caenorhabditis elegans. Nature. 391:806-11.
Miyagishi, M., and K. Taira. 2002. U6 promoter-driven siRNAs with four uridine 3′ overhangs efficiently suppress targeted gene expression in mammalian cells. Nat. Biotechnol. 20:497-500.
Silva, J. M., M. Z. Li, K. Chang, W. Ge, M. C. Golding, R. J. Rickles, D. Siolas, G. Hu, P. J. Paddison, M. R. Schlabach, N. Sheth, J. Bradshaw, J. Burchard, A. Kulkarni, G. Cavet, R. Sachidanandam, W. R. McCombie, M. A. Cleary, S. J. Elledge, and G. J. Hannon. 2005. Second-generation shRNA libraries covering the mouse and human genomes. Nat. Genet. 37:1281-8.
Sui, G., C. Soohoo, B. Affar el, F. Gay, Y. Shi, W. C. Forrester, and Y. Shi. 2002. A DNA vector-based RNAi technology to suppress gene expression in mammalian cells. Proc Natl Acad Sci USA. 99:5515-20.
Verma, S., and F. Eckstein. 1998. Modified oligonucleotides: synthesis and strategy for users. Annu Rev Biochem. 67:99-134.
Yu, J. Y., S. L. DeRuiter, and D. L. Turner. 2002. RNA interference by expression of short-interfering RNAs and hairpin RNAs in mammalian cells. Proc Natl Acad Sci USA. 99:6047-52.

1. A composition useful as transfection agent, comprising polyamines modified by aromatic amino acids and small double-strand or single-strand RNA active for RNA interference.

2. The composition according to claim 1, wherein said polyamines comprise branched or linear polyethylenimine, polyallylamine, dendrimers, polyaminoester, polylysine, polyhistidine, polyarginine, polyornithine or chitosan.

3. The composition according to claim 2, wherein said polyamines are selected in the group comprising linear polyethylenimine (LPET), polyallylamine (PAA) and polylysine (PLL).

4. The composition according to claim 1, wherein the molecular weight of said polyamines is above 400 Da.

5. The composition according to claim 3, wherein the polyamines are selected in the group comprising linear polyethylenimine of 2 KD to 220 KD, polyallylamine of 10 KD to 70 KD and polylysine of 1 KD to 300 KD.

6. The composition according to claim 1, wherein the aromatic amino acids used to modify the polyamines are selected in the group comprising tyrosine, tryptophan and phenylalanine or the derivatives thereof.

7. The composition according to claim 6, wherein said aromatic amino acids are tryptophan and/or tyrosine.

8. The composition according to claim 1, wherein the RNA is normal or modified, the modification groups being for example 2′-Fluo, 2′-Methoxy, phosphorothioate, LNA or morpholino.

9. The composition according to claim 1, wherein the RNA is double stranded or single stranded antisens siRNA or mixtures of single stranded sens/antisens siRNA.

10. The composition according to claim 8, wherein the siRNA has 15-30 mers.

11. The composition according to claim 1, comprising polyamines modified by aromatic amino acids such as above defined and double stranded or single stranded siRNA in an isotonic medium, for example NaCl, glucose, a buffer.

12. The composition according to claim 8, wherein the concentration of siRNA varies from picomolar to micromolar.

13. The composition according to claim 1, further comprising one or several additives such as PEG, PVA, saccharide, polysaccharide, peptide, protein, vitamins.

14. The composition according to claim 1, further comprising plasmid DNA expressing active RNAs for RNAi or encoding a transgene.

15. The composition of claim 14, wherein the transgene is targeted by the said small RNA.

16. The composition of claim 14, wherein said plasmid expresses siRNA, shRNA or microRNA-adapted short hairpin RNA comprising polyamines modified by aromatic amino acids, particularly tyrosine and derivatives thereof, particularly linear polyethylenimine with tyrosine and derivatives thereof, arid useful as DNA vector transfection agent wherein said plasmid expresses siRNA, shRNA or microRNA-adapted short hairpin RNA.

17. A method for synthesizing the polyamines modified by aromatic amino acids of the composition according to claim 1, comprising the use of super-ester of aromatic amino acids activated by Dimethoxytriazine-N-methylmOrphOlium (DMTMM) in the presence of the polyamines.

18. The method according to claim 17, wherein the synthesis of the polyamines is carried out in a basic buffer such as a borate buffer 200 mM, pH=7.5-9 or in an aqueous medium in the presence of a base or a water/alcohol mixture.

19. The method according to claim 17, wherein the percentage of modification of polyamines by aromatic amino acids in said composition varies from 0.01% to 100%, particularly of 15% to 50%.

20. A method for in vitro, ex-vivo transferring siRNA or siRNA and plasmid DNA, comprising using a composition according to claim 1.

21. The method according to claim 20, wherein said in vitro transfer of siRNA is carried out in a medium culture containing adherent cells or cells in suspension.

22. The method according to claim 21, wherein said medium is a normal medium or synthetic medium.


SEQUENCE LISTING (8 sequences)

SEQ ID NO: 1 - DNA, 23 bp, Artificial sequences
  Oligonucleotides
cuuacgcugaguacuucgadtdt

SEQ ID NO: 2 - DNA, 23 bp, Artificial sequences
  Oligonucleotides
dtdtgaaugcgacucaugaagcu

SEQ ID NO: 3 - DNA, 23 bp, Artificial sequences
  Oligonucleotides
cguacgcggaauacuucgadtdt

SEQ ID NO: 4 - DNA, 23 bp, Artificial sequences
  Oligonucleotides
dtdtgcaugcgccuuaugaagcu

SEQ ID NO: 5 - DNA, 23 bp, Artificial sequences
  Oligonucleotides
gcaccacuaguugguugucdtdt

SEQ ID NO: 6 - DNA, 23 bp, Artificial sequences
  Oligonucleotides
dtdtcguggugaucaaccaacag

SEQ ID NO: 7 - DNA, 23 bp, Artificial sequences
  Oligonucleotides
cuggacuuccagaagaacadtdt

SEQ ID NO: 8 - DNA, 23 bp, Artificial sequences
  Oligonucleotides
dtdtgaccugaaggucuucuugu